SEQUENCE LISTING (5 sequences)

SEQ ID NO: 1 - protein, 32 aa, Listeria monocytogenes
KENSISSMAPPASPPASPKTPIEKKHADEIDK

SEQ ID NO: 2 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 4 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 9 aa, Artificial Sequence
  E7 peptide
RAHYNIVTF

(19) United States
(12) Patent Application Publication
(10) Pub. No.: US 2017/0080064 A1
(43) Pub. Date: Mar. 23, 2017

(54) METHODS AND COMPOSITIONS FOR INCREASING A T-EFFECTOR CELL TO REGULATORY T CELL RATIO

(71) Applicants: ADVAXIS, INS., East Princeton, NJ (US); NATIONAL INSTITUTES OF HEALTH (NIH), U.S. DEPT. OF HEALTH AND HUMANS SERVICES (DHHS), U.S. GOVERNMEN, Bethesda, MD (US)

(72) Inventors: Robert Petit, Newtown (Wrightstown), PA (US); Anu Wallecha, Yardley, PA (US); Zhisong Chen, Potomac, MD (US); Jay A Berzofsky, Bethesda, MD (US); Samir Khleif, Silver Spring, MD (US)

(21) Appl. No.: 15/123,468

(22) PCT Filed: Mar. 5, 2015
(86) PCT No.: PCT/US2015/018915

§ 371 Date: Sep. 2, 2016

Related U.S. Application Data
(60) Provisional application No. 61/948,453, filed on Mar. 5, 2014.

(51) Int. Cl. A61K 39/00

(52) U.S. Cl. CPC A61K 39/0011; A61K 2039/6037; A61K 2039/572; A61K 2039/523; C12N 2710/20034; C12N 2710/20022; A61K 2039/585; C12N 2710/20071

(57) ABSTRACT

The present invention is directed to methods for increasing T-cell effector cell to regulatory T cell ratio. The invention is further directed to methods of treating, protecting against, and inducing an immune response against a tumor, comprising the step of administering to a subject a recombinant Listeria strain, comprising a fusion peptide that comprises an LLO fragment and tumor-associated antigen.

### GOVERNMENT INTEREST

This invention was supported, in part, by a Cooperative Research and Development Agreement (CRADA) #02648. The U.S. government may have certain rights in the invention.

### FIELD OF INVENTION

The present invention is directed to methods for increasing T-cell effector cell to regulatory T cell ratio. The invention is further directed to methods of treating, protecting against, and inducing an immune response against a tumor, comprising the step of administering to a subject a recombinant Listeria strain, comprising a fusion peptide that comprises an LLO fragment and tumor-associated antigen.

### BACKGROUND OF THE INVENTION

Listeria monocytogenes (Lm) is a Gram-positive facultative intracellular pathogen that causes listeriolysis. Once invading a host cell, Lm can escape from the phagolysosome through production of a pore-forming protein listeriolysin O (LLO) to lyse the vascular membrane, allowing it to enter the cytoplasm, where it replicates and spreads to adjacent cells based on the mobility of actin-polymerizing protein (ActA). In the cytoplasm, Lm-secreting proteins are degraded by the proteasome and processed into peptides that associate with MHC class I molecules in the endoplasmic reticulum. This unique characteristic makes it a very attractive cancer vaccine vector in that tumor antigen can be presented with MHC class I molecules to activate tumor-specific cytotoxic T lymphocytes (CTLs).

While prophylactic HPV vaccines have been shown effective to protect from HPV infection and from development of cervical intraepithelial neoplasia (CIN), a therapeutic vaccine for advanced cervical cancer patients is still being developed. Progress has been made on construction of an Lm-LLO-E7 vaccine, a live-attenuated Lm-based vaccine producing and secreting a fusion protein consisting of a truncated LLO and full length of E7 antigen. It was shown that Lm-LLO-E7 was able to induce complete regression of established HPV-immortalizing TC-1 tumors in mice. The anti-tumor activity induced by Lm-LLO-E7 was critically mediated by CD8+ T cells, as depletion of these totally abrogated the inhibition of tumor growth and it was also observed that the Lm-LLO-E7 vaccine decreased regulatory T cells (Tregs). Tregs, identified as CD4+FoxP3+(or CD4+CD25+ when first discovered) T cells, are a small population that suppresses immunity.

It is conceivable that Lm-LLO-E7-induced Treg decrease may contribute to its anti-tumor effect, but how an Lm-LLO-E7 vaccine induces Treg decrease remains unclear yet. There's a need for identifying the mechanism by which Lm-LLO-E7 causes Treg reduction in order to further improve its anti-tumor efficacy by developing novel therapeutic strategies to manipulate Tregs.

The present invention provides an effective and safe immunotherapy detailing how the immunosuppresive effects of regulatory T cells can be overcome in order to trigger helpful immune responses. This immunotherapy employs the use of an attenuated recombinant Listeria comprising a mutation in the endogenous dal, dat, and actA and episomally expressing a N-terminal truncated LLO.

### SUMMARY OF THE INVENTION

In one embodiment, the invention relates to a method of eliciting an anti-tumor T cell response in a subject having said tumor, comprising the step of administering to said subject a recombinant Listeria strain comprising a recombinant nucleic acid, said nucleic acid molecule comprising a first open reading frame encoding a recombinant polypeptide and a second open reading frame second open reading frame encoding a metabolic, wherein said recombinant polypeptide comprises a truncated LLO protein fused to a heterologous antigen or fragment thereof, wherein said Listeria comprises a mutation in the endogenous alanine racemase gene (dal), D-amino acid transferase gene (dat), and actA genes, wherein said T-cell response comprises increasing a ratio of T effector cells to regulatory T cells (Tregs) in the spleen of said subject. In another embodiment, eliciting an anti-tumor T cell response in a subject having a tumor or cancer allows treating said tumor or cancer in said subject. In another embodiment, eliciting an anti-tumor T cell response in a subject having a tumor or cancer prevents the establishment of metastases in said subject.

In another embodiment, the invention relates to a method for increasing the ratio of T effector cells to regulatory T cells (Tregs) in the spleen of a subject, the method comprising the step of administering to said subject a recombinant Listeria strain comprising a recombinant nucleic acid encoding a truncated LLO protein, wherein said Listeria comprises a mutation in the endogenous alanine racemase gene (dal), D-amino acid transferase gene (dat), and actA genes, wherein said T-cell response comprises increasing a ratio of T effector cells to regulatory T cells (Tregs).

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. LmddA-LLO-E7 induces regression of established TC-1 tumors accompanying with Treg frequency decrease. C57BL6 mice were inoculated s.c. with 1×10⁵ TC-1 tumor cells each, and were immunized i.p. with 0.1 LD50 LmddA-LLO-E7 (1×10⁸ CFU), Lm-E7 (1×10⁶ CFU), or LmddA-LLO (1×10⁸ CFU) in PBS (100 μl) on day 10 and day 17 post tumor challenge. Tumor was measured twice a week using an electronic caliper. Tumor volume was calculated by the formula: length×width×width/2. Mice were sacrificed when tumor diameter reached approximately 2.0 cm or on day 24 for Flow cytometric analysis. (A) Average tumor volume from day 10 to day 24. (B) Tumor volume on day 24. (C) Survival percentage. (D) Flow cytometric profile of CD4+FoxP3+ T cells out of CD4+ T cells. (E) Percentage of CD4+FoxP3+ T cells out of CD4+ T cells in the spleen. (F) Ratio of CD4+FoxP3+ T cells to CD8+ T cells in the spleen. (G) Percentage of CD4+FoxP3+ T cells out of CD4+ T cells in the tumor. (H) Ratio of CD4+FoxP3+ T cells to CD8+ T cells in the tumor. Data are presented as Mean±SEM. *P<0.05, **P<0.01, and ***P<0.001 (Mann-Whitney test). Data are from 3 independent experiments (A and B) and are representative of 3 independent experiments (C-H).

FIG. 2. LmddA-LLO-E7 induces regression of established TC-1 tumors. C57BL6 mice were inoculated s.c. with 1×10⁵ TC-1 tumor cells each, and were immunized i.p. with 0.1 LD50 LmddA-LLO-E7 (1×10⁸ CFU), Lm-E7 (1×10⁶ CFU), or LmddA-LLO (1×10⁸ CFU) in PBS (100 μl) on day 10 and day 17 post tumor challenge. Tumor was measured twice a week using an electronic caliper. Tumor volume was calculated by the formula: length×width×width/2. (A) PBS. (B) LmddA. (C) Lm-E7. (D) LmddA-LLO-E7. Data are from 3 independent experiments.

FIG. 3. LmddA-LLO-E7 and Lm-E7 induce similar E7-specific CD8+ T cell response. C57BL6 mice were inoculated s.c. with 1×10⁵ TC-1 tumor cells each, and were immunized i.p. with 0.1 LD50 LmddA-LLO-E7 (1×10⁸ CFU), LmddA-LLO (1×10⁸ CFU), LmddA (1×10⁸ CFU), Lm-E7 (1×10⁶ CFU), or 0.5 LD50 wild-type Lm 10403S (1×10⁴ CFU) in PBS (100 μl) on day 10 and day 17 post tumor challenge. Mice were sacrificed at day 24 and lymphocytes isolated from the spleen and tumor were analyzed by Flow cytometry. A. Flow cytometric prolife of H-2D^{b} E7 tetramer+CD8+ T cells out of CD8+ T cells in the spleen and tumor. (B and C) Percentage of H-2D^{b} E7 tetramer+CD8+ T cells out of CD8+ T cells in the spleen (B) and tumor (C). (D and E) H-2D^{b} E7 tetramer+CD8+ T cell number per mouse spleen (D) and per million tumor cells (E). n=3-10. Data are representative of 3 independent experiments.

FIG. 4. L. monocytogenes is sufficient to induce decrease of Treg frequency. C57BL6 mice were inoculated s.c. with 1×10⁵ TC-1 tumor cells each, and were immunized i.p. with 0.1 LD50 LmddA (1×10⁸ CFU) or 0.5 LD50 wild-type Lm 10403S (1×10⁴ CFU) in PBS (100 μl) on day 10 and day 17 post tumor challenge. Mice were sacrificed at day 24 and lymphocytes isolated from the spleen and tumor were analyzed by Flow cytometry. (A) Flow cytometric profile of CD4+FoxP3+ T cells out of CD4⁺ T cells. (B) Percentage of CD4+FoxP3+ T cells out of CD4+ T cells in the spleen. (C) Ratio of CD4+FoxP3+ T cells to CD8+ T cells in the spleen. (D) Percentage of CD4+FoxP3+ T cells out of CD4+ T cells in the tumor. (E) Ratio of CD4+FoxP3+ T cells to CD8+ T cells in the tumor. Data are presented as Mean±SEM. *P<0.05, **P<0.01, and ***P<0.001 (Mann-Whitney test). Data are representative of 3 independent experiments.

FIG. 5. L. monocytogenes decreases Treg frequency by preferentially inducing CD4+FoxP3− T cell and CD8+ T cell expansion. C57BL6 mice were inoculated s.c. with 1×10⁵ TC-1 tumor cells each, and were immunized i.p. with 0.1 LD50 LmddA-LLO-E7 (1×10⁸ CFU), LmddA-LLO (1×10⁸ CFU), LmddA (1×10⁸ CFU), Lm-E7 (1×10⁶ CFU), or 0.5 LD50 wild-type Lm 10403S (1×10⁴ CFU) in PBS (100 μl) on day 10 and day 17 post tumor challenge. Mice were sacrificed at day 24 and lymphocytes isolated from the tumor were analyzed by Flow cytometry. Data are presented as (Mean±SEM). n=3-10. *P<0.05, **P<0.01 (Mann-Whitney test). Data are representative of 3 independent experiments.

FIG. 6. L. monocytogenes-induced expansion of CD4+FoxP3− T cells and CD8+ T cells is dependent on and mediated by LLO. C57BL6 mice were injected i.p. with 1×10⁴ CFU 10403S, Δhly, Δhly::pfo, or hly::Tn917-lac (pAM401-hly) in PBS (100 μl). Mice were sacrificed on day 7 post injection and lymphocytes isolated from the spleen were analyzed by Flow cytometry. (A) T cell number in the spleen. (B) Flow cytometric prolife of CD4+FoxP3+ T cells out of CD4+ T cells. (C) Percentage of CD4+FoxP3+ T cells out of CD4+ T cells. (D) Ratio of CD4+FoxP3+ T cells to CD8+ T cells. *P<0.05 (Mann-Whitney test). Data are representative of 3 independent experiments.

FIG. 7. Episomal expression of a truncated LLO in LmddA induces expansion of CD4+FoxP3− T cells and CD8+ T cells to a higher level. C57BL6 mice were injected i.p. with 1×10⁸ CFU LmddA or LmddA-LLO in PBS (100 μl). Mice were sacrificed on day 7 post injection and lymphocytes isolated from the spleen were analyzed by Flow cytometry. (A) T cell number in the spleen. (B) Flow cytometric prolife of CD4+FoxP3+ T cells out of CD4+ T cells. (C) Percentage of CD4+FoxP3+ T cells out of CD4+ T cells. (D) Ratio of CD4+FoxP3+ T cells to CD8+ T cells. (E) Flow cytometric prolife of Ki-67+ T cells. (F) Percentage of Ki-67+ T cells. (G) Fluorescent intensity of Ki-67+ T cells. Data are presented as Mean±SEM. *P<0.05, **P<0.01, and ***P<0.001 (Mann-Whitney test). Data are representative of 3 independent experiments.

FIG. 8. Combination of Lm-E7 and LmddA-LLO induces regression of established TC-1 tumors. C57BL/6 mice were inoculated s.c. with 1×10⁵ TC-1 tumor cells each, and were immunized i.p. with 0.05 LD50 Lm-E7 (5×10⁵ CFU), 0.05 LD50 LmddA-LLO (5×10⁷ CFU), 0.05 LD50 Lm-E7 plus 0.05 LD50 LmddA-LLO in PBS (100 μl) on day 10 and day 17 post tumor challenge. Tumor was measured twice a week using an electronic caliper and tumor volume was calculated by the formula: length×width×width/2. Mice were observed for survival or sacrificed on day 24 and lymphocytes isolated from the spleen were analyzed by Flow cytometry. (A) Average tumor volume from day 10 to day 24. (B) Tumor volume on day 24. (C) Survival percentage. (D) T cell number in the spleen. (E) Flow cytometric prolife of CD4+FoxP3+ T cells out of CD4+ T cells. (F) Percentage of CD4+FoxP3+ T cells out of CD4+ T cells. (G) Ratio of CD4+FoxP3+ T cells to CD8+ T cells. Data are presented as Mean±SEM. *P<0.05, **P<0.01, and ***P<0.001 (Mann-Whitney test). Data are representative of 2 independent experiments.

FIG. 9. Adoptive transfer of Tregs compromises the anti-tumor efficacy of LmddA-LLO-E7 against established TC-1 tumors. C57BL6 mice (11 weeks old) were injected s.c. with 1×10⁵ TC-1 tumor cells each, and i.v. with CD4+CD25+ Tregs (1×10⁶ cells/each) on day 9 post tumor challenge. Mice were immunized i.p. with 0.1 LD50 LmddA-LLO-E7 (1×10⁸ CFU) in PBS (100 μl) on day 10 and day 17 post tumor challenge. Tumor was measured twice a week using an electronic caliper and tumor volume was calculated by the formula: length×width×width/2. Mice were sacrificed on day 24 and lymphocytes isolated from the spleen were analyzed by Flow cytometry. (A) Average tumor volume from day 10 to day 24. (B) Tumor volume on day 24. (C) Flow cytometric prolife of CD4+FoxP3+ T cells out of CD4+ T cells. (D) Percentage of CD4+FoxP3+ T cells out of CD4+ T cells in the spleen. (E) Percentage of CD4+FoxP3+ T cells out of CD4+ T cells in the tumor. (F) T cell number in the spleen. (G) T cell number per million tumor cells. Data are presented as Mean±SEM. *P<0.05, **P<0.01, and ***P<0.001 (Mann-Whitney test). Data are representative of 2 independent experiments.

FIG. 10. LmddA does not augment Lm-E7 anti-tumor activity. C57BL/6 mice were inoculated s.c. with 1×10⁵ TC-1 tumor cells each, and were immunized i.p. with 0.05 LD50 Lm-E7 (5×10⁵ CFU), 0.05 LD50 LmddA (5×10⁷ CFU), or 0.05 LD50 Lm-E7 plus 0.05 LD50 LmddA in PBS (100 μl) on day 10 and day 17 post tumor challenge. Tumor was measured using an electronic caliper and tumor volume was calculated by the formula: length×width×width/2. Shown are tumor volumes on day 24. Data are presented as Mean±SEM.

### DETAILED DESCRIPTION OF THE INVENTION

This invention provides in one aspect a recombinant Listeria vaccine vector comprising a recombinant nucleic acid encoding a recombinant polypeptide, wherein the recombinant polypeptide comprises a non-hemolytic N-terminal Listeriolysin (LLO) fused to a heterologous antigen, wherein said Listeria comprises a mutation in the endogenous dal/dat and actA genes, wherein said T-cell response comprises increasing a ratio of T effector cells to regulatory T cells (Tregs) in the spleen of said subject.

This invention provides in another aspect a method of eliciting an anti-tumor T cell response in a subject having said tumor, comprising the step of administering to said subject a recombinant Listeria strain comprising a recombinant nucleic acid, said nucleic acid molecule comprising a first open reading frame encoding a recombinant polypeptide and a second open reading frame second open reading frame encoding a metabolic, wherein said recombinant polypeptide comprises a truncated LLO protein fused to a heterologous antigen or fragment thereof, wherein said Listeria comprises a mutation in the endogenous alanine racemase gene (dal), D-amino acid transferase gene (dat), and actA genes, wherein said T-cell response comprises increasing a ratio of T effector cells to regulatory T cells (Tregs) in the spleen of said subject. In another embodiment, eliciting an anti-tumor T cell response in a subject having a tumor or cancer allows treating said tumor or cancer in said subject. In another embodiment, eliciting an anti-tumor T cell response in a subject having a tumor or cancer prevents the establishment of metastases in said subject.

In another embodiment, the heterologous antigen is a tumor-associated antigen.

In one embodiment, increasing a ratio of T effector cells to regulatory T cells (Tregs) in the spleen of said subject allows for a more profound anti-tumor response in said subject.

In one embodiment, the recombinant Listeria strain provided herein lacks antibiotic resistance genes.

In a further aspect, this invention provides a method for increasing the ratio of T effector cells to regulatory T cells (Tregs) in the spleen of a subject, the method comprising the step of administering to said subject a recombinant Listeria strain comprising a recombinant nucleic acid encoding a truncated LLO protein, wherein said Listeria comprises a mutation in the endogenous alanine racemase gene (dal), D-amino acid transferase gene (dat), and actA genes, wherein said T-cell response comprises increasing a ratio of T effector cells to regulatory T cells (Tregs).

In one embodiment, the recombinant Listeria provided herein is capable of escaping the phagolysosome.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In one embodiment, the present invention provides methods of treating, protecting against, and inducing an immune response against a tumor or a cancer, comprising the step of administering to a subject the recombinant Listeria strain provided herein.

In one embodiment, the present invention provides a method of treating a tumor or cancer in a human subject, comprising the step of administering to the subject the recombinant Listeria strain provided herein, the recombinant Listeria strain comprising a recombinant polypeptide comprising an N-terminal fragment of an LLO protein and tumor-associated antigen, whereby the recombinant Listeria strain induces an immune response against the tumor-associated antigen, thereby treating a tumor or cancer in a human subject. In another embodiment, the immune response is an T-cell response. In another embodiment, the T-cell response is a CD4+FoxP3− T cell response. In another embodiment, the T-cell response is a CD8+ T cell response. In another embodiment, the T-cell response is a CD4+FoxP3- and CD8+ T cell response.

In another embodiment, the present invention provides a method of protecting a subject against a tumor or cancer, comprising the step of administering to the subject the recombinant Listeria strain provided herein. In another ambodiment, the present invention provides a method of inducing regression of a tumor in a subject, comprising the step of administering to the subject the recombinant Listeria strain provided herein. In another embodiment, the present invention provides a method of reducing the incidence or relapse of a tumor or cancer, comprising the step of administering to the subject the recombinant Listeria strain provided herein. In another embodiment, the present invention provides a method of suppressing the formation of a tumor in a subject, comprising the step of administering to the subject the recombinant Listeria strain provided herein. In another embodiment, the present invention provides a method of inducing a remission of a cancer in a subject, comprising the step of administering to the subject a recombinant Listeria strain provided herein.

In one embodiment, the Listeria genome comprises a deletion of the endogenous ActA gene, which in one embodiment is a virulence factor. In one embodiment, such a deletion provides a more attenuated and thus safer Listeria strain for human use. According to this embodiment, the antigenic polypeptide is integrated in frame with LLO in the Listeria chromosome. In another embodiment, the integrated nucleic acid molecule is integrated into the ActA locus. In another embodiment, the chromosomal nucleic acid encoding ActA is replaced by a nucleic acid molecule encoding an antigen.

In one embodiment, the nucleic acid molecule provided herein comprises a first open reading frame encoding recombinant polypeptide comprising a heterologous antigen or fragment thereof. In another embodiment, the recombinant polypeptide further comprises a N-terminal LLO fused to the heterologous antigen. In another embodiment, the nucleic acid molecule provided herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains provided herein comprise a mutation or a deletion in the genomic dal/dat genes. In another embodiment, the Listeria lack dal/dat genes.

In another embodiment, a nucleic acid molecule of the methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence. Each possibility represents a separate embodiment of the present invention.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the present invention.

In another embodiment, the recombinant Listeria is an attenuated auxotrophic strain.

In one embodiment the attenuated strain is Lm dal(−)dat(−) (Lmdd). In another embodiment, the attenuated strains is Lm dal(−)dat(−)ΔactA (LmddA). LmddA is based on a Listeria vaccine vector which is attenuated due to the deletion of virulence gene actA and retains the plasmid for a desired heterologous antigen or truncated LLO expression in vivo and in vitro by complementation of dal gene.

In another embodiment the attenuated strain is Lmdda. In another embodiment, the Listeria strains provided herein comprise a mutation or a deletion in the genomic dal/dat/actA genes. In another embodiment, the Listeria lack dal/dat/actA genes. In another embodiment, the attenuated strain is LmΔactA. In another embodiment, the attenuated strain is LmΔPrfA. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain used in the invention is a Listeria strain that expresses a non-hemolytic LLO.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as provided herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, said metabolic enzyme is an amino acid metabolism enzyme. In another embodiment, said metabolic enzyme catalyzes a formation of an amino acid used for a cell wall synthesis in said recombinant Listeria strain. In another embodiment, said metabolic enzyme is an alanine racemase enzyme. In another embodiment, said metabolic enzyme is a D-amino acid transferase enzyme. Each possibility represents a separate embodiment of the methods and compositions as provided herein.

In one embodiment, said auxotrophic Listeria strain comprises an episomal expression vector comprising a metabolic enzyme that complements the auxotrophy of said auxotrophic Listeria strain. In another embodiment, the construct is contained in the Listeria strain in an episomal fashion. In another embodiment, the foreign antigen is expressed from a vector harbored by the recombinant Listeria strain. In another embodiment, said episomal expression vector lacks an antibiotic resistance marker. In one embodiment, an antigen of the methods and compositions as provided herein is fused to an polypeptide comprising a PEST sequence. In another embodiment, said polypeptide comprising a PEST sequence is a truncated LLO. In another embodiment, said polypeptide comprising a PEST sequence is ActA.

In another embodiment, the Listeria strain is deficient in an AA metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is filI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, provided herein is a nucleic acid molecule that is used to transform the Listeria in order to arrive at a recombinant Listeria. In another embodiment, the nucleic acid provided herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria genome. In another embodiment, the virulence gene is deleted in the recombinant Listeria genome. In another embodiment, the virulence gene is truncated in the recombinant Listeria genome. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, a prfA gene or a combination thereof It is to be understood by a skilled artisan, that the virulence gene can be any gene known in the art to be associated with virulence in the recombinant Listeria.

In yet another embodiment the Listeria strain is an inlA mutant, an inlB mutant, an inlC mutant, an inlJ mutant, prfA mutant, actA mutant, a prfA mutant, a plcB deletion mutant, a double mutant in both the plcA and plcB genes, or a double mutant in the actA and inlB genes. In another embodiment, the Listeria comprise a deletion or mutation of these genes individually or in combination. In another embodiment, the Listeria provided herein lack each one of genes. In another embodiment, the Listeria provided herein lack at least one and up to ten of any gene provided herein, including the actA, prfA, and dal/dat genes. In one embodiment, the live attenuated Listeria is a recombinant Listeria. In another embodiment, the recombinant Listeria comprises a mutation or a deletion of a genomic internalin C (inlC) gene. In another embodiment, the recombinant Listeria comprises a mutation or a deletion of a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a vaccine backbone. Each possibility represents a separate embodiment of the present invention.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome. Each possibility represents a separate embodiment of the present invention.

In one embodiment, in order to select for an auxotrophic bacteria comprising a plasmid encoding a metabolic enzyme or a complementing gene provided herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with this invention.

In one embodiment, the N-terminal LLO protein fragment and heterologous antigen are, in another embodiment, fused directly to one another. In another embodiment, the genes encoding the N-terminal LLO protein fragment and heterologous antigen are fused directly to one another. In another embodiment, the N-terminal LLO protein fragment and heterologous antigen are attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is the N-terminal-most portion of the fusion protein. Each possibility represents a separate embodiment of the present invention. As provided herein, recombinant Listeria strains expressing LLO unexpectedly increase CD4+FoxP3− T cell and CD8+ T cell number in the spleen to a level higher than a recombinant Listeria strain not expressing truncated LLO (Example 5), thereby demonstrating that expansion of CD4+FoxP3− T cells and CD8+ T cells is directly mediated by LLO (Example 4). As further provided herein, the recombinant Listeria episomally expressing a truncated LLO unexpectedly increases the ratio of CD4+FoxP3− T cell and CD8+ T cell to CD4+FoxP3+ T cell (regulatory T cells or Tregs) by inducing the expansion of CD4+FoxP3-T cell and CD8+ T, without reducing the number to Tregs, thereby decreasing the frequency of Tregs in a proportionate manner. As further provided herein, the recombinant Listeria expressing HPV-E7 in the context of a fusion protein with LLO preferentially induces CD4+FoxP3− T cell and CD8+ T cell expansion, which enhances the vaccine's anti-tumor activity and upregulates the expression of chemokine receptors CCR5 and CXCR3 on CD4+FoxP3− T cells and CD8+ T cells, but not on CD4+FoxP3+ T cells showing that CCR5 and CXCR3 are crucial for Th1 and CD8+ T cell trafficking (see example 7).

In one embodiment, a recombinant Listeria strain provided herein comprises a recombinant polypeptide. In another embodiment, a recombinant Listeria strain provided herein expresses a recombinant polypeptide. In another embodiment, the recombinant Listeria strain comprises a plasmid that encodes the recombinant polypeptide. In another embodiment, the recombinant Listeria strain comprises a recombinant nucleic acid encoding the recombinant polypeptide provided herein. In another embodiment, a plasmid provided herein is an episomal plasmid that does not integrate into said recombinant Listeria strain's chromosome. In another embodiment, the plasmid is an integrative plasmid that integrates into said Listeria strain's chromosome. In another embodiment, the plasmid is a multicopy plasmid.

In another embodiment, a method of the present invention further comprises boosting the subject with a immunogenic composition comprising an attenuated Listeria strain provided herein. In another embodiment, a method of the present invention comprises the step of administering a booster dose of the immunogenic composition comprising the attenuated Listeria strain provided herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria provided herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HW, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In another embodiment, a recombinant Listeria strain is used in the booster inoculation. In another embodiment, the recombinant Listeria strain used in the booster inoculation is the same as the strain used in the initial “priming” inoculation. In another embodiment, the booster strain is different from the priming strain. In another embodiment, the recombinant immune checkpoint inhibitor used in the booster inoculation is the same as the inhibitor used in the initial “priming” inoculation. In another embodiment, the booster inhibitor is different from the priming inhibitor. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In one embodiment, a treatment protocol of the present invention is therapeutic. In another embodiment, the protocol is prophylactic. In another embodiment, the compositions of the present invention are used to protect people at risk for cancer such as breast cancer or other types of tumors because of familial genetics or other circumstances that predispose them to these types of ailments as will be understood by a skilled artisan. In another embodiment, the compositions provided herein are used as a cancer immunotherapy after debulking of tumor growth by surgery, conventional chemotherapy or radiation treatment. Following such treatments, the vaccines of the present invention are administered so that the CTL response to the tumor antigen of the vaccine destroys remaining metastases and prolongs remission from a cancer. In another embodiment, vaccines of the present invention are used to effect the growth of previously established tumors and to kill existing tumor cells. Each possibility represents a separate embodiment of the present invention.

In one embodiment, the method provided herein comprises the step of boosting a human subject with a recombinant Listeria strain of the present invention. In another embodiment, the method further comprises the step of boosting the human subject with an immunogenic composition comprising an E7 antigen. In another embodiment, the method further comprises the step of boosting the human subject with an immunogenic composition that directs a cell of the subject to express an E7 antigen. Each possibility represents a separate embodiment of the present invention.

“Boosting” refers, in another embodiment, to administration of an additional vaccine dose or additional therapy dos to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered. Each possibility represents a separate embodiment of the present invention.

In one embodiment, the method provided herein comprises the step of co-administering the recombinant Listeria with an additional therapy. In another embodiment, the additional therapy is surgery, chemotherapy, an immunotherapy or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the antibody therapy is an anti-PD1, anti-CTLA4. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in US Patent Serial No. 6,991,785, incorporated by reference herein.

In some embodiments, the term “antibody” refers to intact molecules as well as functional fragments thereof, such as Fab, F(ab′)2, and Fv that are capable of specifically interacting with a desired target as described herein, for example, binding to phagocytic cells. In some embodiments, the antibody fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

In some embodiments, the antibody fragments may be prepared by proteolytic hydrolysis of the antibody or by expression in E. coli or mammalian cells (e.g. Chinese hamster ovary cell culture or other protein expression systems) of DNA encoding the fragment.

Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained to therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In some embodiments, the antibodies or fragments as described herein may comprise “humanized forms” of antibodies. In some embodiments, the term “humanized forms of antibodies” refers to non-human (e.g. murine) antibodies, which are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The term “epitope” or antigenic determinant” refers to a site on an antigen to which an immunoglobulin or antibody, or fragment thereof, specifically binds. Epitopes can be formed both from contiguous amino acids or noncontiguous amino acids juxtaposed by tertiary folding of a protein. Epitopes formed from continuous aminio acis are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, or 15 amino acids in a unique spatial conformation. Methods of determining spatial conformation of epitopes include, for example, x-ray crystallography and 2-dimensional nuclear magnetic resonance.

In one embodiment, compositions of this invention comprise a therapeutic or immunomodulating monoclonal antibody. In another embodiment, a composition of this invention comprises an Lm strain and a therapeutic or immunomodulating monoclonal antibody. In another embodiment, a composition of this invention comprises a therapeutic or immunomodulating monoclonal antibody, wherein the composition does not include a Listeria strain provided herein.

In one embodiment, the heterologous antigen is a tumor-associated antigen. In another embodiment, the tumor-associated antigen is HPV-E7. In another embodiment, the antigen is HPV-E6. In another embodiment, the antigen is Her-2. In another embodiment, the antigen is NY-ESO-1. In another embodiment, the antigen is telomerase. In another embodiment, the antigen is SCCE. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from E7, E6, Her-2, NY-ESO-1, telomerase, SCCE, WT-1, HIV-1 Gag, Proteinase 3, Tyrosinase related protein 2, PSA (prostate-specific antigen). In another embodiment, the antigen is a tumor-associated antigen. In another embodiment, the antigen is an infectious disease antigen.

In another embodiment, the tumor-associated antigen is an angiogenic antigen. In another embodiment, the angiogenic antigen is expressed on both activated pericytes and pericytes in tumor angiogeneic vasculature, which in another embodiment, is associated with neovascularization in vivo. In another embodiment, the angiogenic antigen is HMW-MAA. In another embodiment, the angiogenic antigen is one known in the art and are provided in WO2010/102140, which is incorporated by reference herein.

In one embodiment, compositions of the present invention induce a strong stimulation of interferon-gamma, which in one embodiment, has anti-angiogenic properties. In one embodiment, a Listeria of the present invention induces a strong stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, compositions of the present invention induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

In other embodiments, an antigen for use in the compositions and methods provided herein is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, mesothelin, EGFRVIII or pSA.

In other embodiments, an antigen for use in the compositions and methods provided herein is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In other embodiments, an antigen for use in the compositions and methods provided herein is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer.

The HPV that is the target of methods of the present invention is, in another embodiment, an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type. Each possibility represents a separate embodiment of the present invention.

In one embodiment, the disease provided herein is an infectious disease, a cancer or a tumor.

In one embodiment, the infectious disease is one caused by, but not limited to, any one of the following pathogens: BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arena viruses (LCM, Junin virus, Machupo virus, Guanarito virus, Lassa Fever), Bunyaviruses (Hantaviruses, Rift Valley Fever), Flaviruses (Dengue), Filo viruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tick borne hemorrhagic fever viruses, Chikungunya virus, Crimean-Congo Hemorrhagic fever virus, Tick borne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein. In another embodiment, an infection occurs following a transplantation when a subject immune system may be compromised.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas.

In one embodiment, the cancer treated by a method of the present invention is breast cancer. In another embodiment, the cancer is a cervical cancer. In another embodiment, the cancer is an HER2 expressing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, it is a hypoxic solid tumor. In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. In another embodiment, the cancer is mesothelioma. Each possibility represents a separate embodiment of the present invention.

In one embodiment, a truncated LLO provided herein comprises a putative PEST amino acid (AA) sequence. In another embodiment, the PEST amino acid sequence is KENSISSMAPPASPPASPKTPIEKKHADEIDK (SEQ ID NO: 1). In another embodiment, fusion of an antigen to other LM PEST AA sequences from Listeria will also enhance immunogenicity of the antigen.

The N-terminal LLO protein fragment of methods and compositions of the present invention comprises, in another embodiment, SEQ ID No: 1. In another embodiment, the fragment comprises an LLO signal peptide. In another embodiment, the fragment comprises SEQ ID No: 2. In another embodiment, the fragment consists approximately of SEQ ID No: 2. In another embodiment, the fragment consists essentially of SEQ ID No: 2. In another embodiment, the fragment corresponds to SEQ ID No: 2. In another embodiment, the fragment is homologous to SEQ ID No: 2. In another embodiment, the fragment is homologous to a fragment of SEQ ID No: 2. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the present invention. In another embodiment, fusion of a heterologous antigen to any ΔLLO, including the PEST AA sequence, SEQ ID NO: 1, enhances cell mediated and anti-tumor immunity of the antigen. Each possibility represents a separate embodiment of the present invention.

The LLO protein utilized to construct vaccines of the present invention has, in another embodiment, the sequence:

MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKSINQNNADIQVVNAISSLTYPGALVKANSELVENQPDVLPVKRDS
LTLSIDLPGMTNQDNKIVVKNATKSNVNNAVNTLVERWNEKYAQAYPNVS
AKIDYDDEMAYSESQLIAKFGTAFKAVNNSLNVNFGAISEGKMQEEIVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDPEGNEIVQI
IKNWSENNKSKLAHFTSSIYLPGNARNINVYAKECTGLAWEWWRTVIDDR
NLPLVKNRNISIWGTTLYPKYSNKVDNPIE (GenBank Accession
No. P13128; SEQ ID NO: 3; nucleic acid sequence is
set forth in GenBank Accession No. X15127).

The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention. Each possibility represents a separate embodiment of the present invention.

In another embodiment, the N-terminal fragment of an LLO protein utilized in compositions and methods of the present invention has the sequence:

(SEQ ID NO: 2)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 4)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In one embodiment, the present invention provides a recombinant protein or polypeptide comprising a listeriolysin O (LLO) protein or a recombinant Listeria expressing the same, wherein said LLO protein comprises a mutation of residues C484, W491, W492, or a combination thereof of the cholesterol-binding domain (CBD) of said LLO protein (see U.S. Pat. No. 8,771,702, which is hereby incorporated by reference herein). In one embodiment, said C484, W491, and W492 residues are residues C484, W491, and W492 of SEQ ID NO: 3, while in another embodiment, they are corresponding residues as can be deduced using sequence alignments, as is known to one of skill in the art. In one embodiment, residues C484, W491, and W492 are mutated. In one embodiment, a mutation is a substitution, in another embodiment, a deletion. In one embodiment, the entire CBD is mutated, while in another embodiment, portions of the CBD are mutated, while in another embodiment, only specific residues within the CBD are mutated.

In another embodiment, “truncated LLO” or “ΔLLO” refers to a non-hemolytic fragment of LLO that comprises a PEST sequence. In another embodiment, the terms refers to an LLO fragment that comprises a PEST domain. In another embodiment, the LLO fragment is an N-terminal LLO fragment. In another embodiment, the LLO fragment is at least 492 amino acids (AA) long. In another embodiment, the LLO fragment is 492-528 AA long. In another embodiment, the non-LLO peptide is 1-50 amino acids long. In another embodiment, the mutated region is 1-50 amino acids long. In another embodiment, the non-LLO peptide is the same length as the mutated region. In another embodiment, the non-LLO peptide is shorter, or in another embodiment, longer, than the mutated region. In another embodiment, the substitution is an inactivating mutation with respect to hemolytic activity. In another embodiment, the recombinant peptide exhibits a reduction in hemolytic activity relative to wild-type LLO. In another embodiment, the recombinant peptide is non-hemolytic. Each possibility represents a separate embodiment of the present invention.

In one embodiment, the present invention provides a recombinant protein or polypeptide comprising a mutated LLO protein or fragment thereof, wherein the mutated LLO protein or fragment thereof contains a substitution of a non-LLO peptide for a mutated region of the mutated LLO protein or fragment thereof, the mutated region comprising a residue selected from C484, W491, and W492.

As provided herein, a mutant LLO protein comprises a substitution of residues C484, W491, and W492 of wild-type 110.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the present invention.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

In another embodiment, a homologous LLO refers to identity to an LLO sequence (e.g. to one of SEQ ID No: 2-4) of greater than 70%. In another embodiment, a homologous LLO refers to identity to one of SEQ ID No: 2-4 of greater than 72%. In another embodiment, a homologous refers to identity to one of SEQ ID No: 2-4 of greater than 75%. In another embodiment, a homologous refers to identity to one of SEQ ID No: 2-4 of greater than 78%. In another embodiment, a homologous refers to identity to one of SEQ ID No: 2-4 of greater than 80%. In another embodiment, a homologous refers to identity to one of SEQ ID No: 2-4 of greater than 82%. In another embodiment, a homologous refers to identity to one of SEQ ID No: 2-4 of greater than 83%. In another embodiment, a homologous refers to identity to one of SEQ ID No: 2-4 of greater than 85%. In another embodiment, a homologous refers to identity to one of SEQ ID No: 2-4 of greater than 87%. In another embodiment, a homologous refers to identity to one of SEQ ID No: 2-4 of greater than 88%. In another embodiment, a homologous refers to identity to one of SEQ ID No: 2-4 of greater than 90%. In another embodiment, a homologous refers to identity to one of SEQ ID No: 2-4 of greater than 92%. In another embodiment, a homologous refers to identity to one of SEQ ID No: 2-4 of greater than 93%. In another embodiment, a homologous refers to identity to one of SEQ ID No: 2-4 of greater than 95%. In another embodiment, a homologous refers to identity to one of SEQ ID No: 2-4 of greater than 96%. In another embodiment, a homologous refers to identity to one of SEQ ID No: 2-4 of greater than 97%. In another embodiment, a homologous refers to identity to one of SEQ ID No: 2-4 of greater than 98%. In another embodiment, a homologous refers to identity to one of SEQ ID No: 2-4 of greater than 99%. In another embodiment, a homologous refers to identity to one of SEQ ID No: 2-4 of 100%. Each possibility represents a separate embodiment of the present invention.

In another embodiment, the term “homology,” when in reference to any nucleic acid sequence provided herein similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-5 of greater than 70%. In another embodiment, “homology” refers to identity to a sequence selected from SEQ ID No: 1-5 of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the construct or nucleic acid molecule provided herein is integrated into the Listerial chromosome using homologous recombination. Techniques for homologous recombination are well known in the art, and are described, for example, in Baloglu S, Boyle S M, et al (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present invention further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain can be complemented. Each possibility represents a separate embodiment of the present invention.

In another embodiment, the construct or nucleic acid molecule is expressed from an episomal or plasmid vector, with a nucleic acid sequence encoding an LLO, PEST or ActA sequence or fragments thereof. In another embodiment, the plasmid is stably maintained in the recombinant Listeria vaccine strain in the absence of antibiotic selection. In another embodiment, the plasmid does not confer antibiotic resistance upon the recombinant Listeria. In another embodiment, the fragment is a functional fragment. In another embodiment, the fragment is an immunogenic fragment.

In another embodiment, an “immunogenic fragment” is one that elicits an immune response when administered to a subject alone or in a vaccine or composition as provided herein. Such a fragment contains, in another embodiment, the necessary epitopes in order to elicit an adaptive immune response.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the present invention.

In another embodiment, the “functional fragment” is an immunogenic fragment and elicits an immune response when administered to a subject alone or in a vaccine composition provided herein. In another embodiment, a functional fragment has biological activity as will be understood by a skilled artisan and as further provided herein.

In another embodiment, the recombinant Listeria strain is administered to the human subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

Each dose and range of doses represents a separate embodiment of the present invention.

In another embodiment, the recombinant polypeptide of methods of the present invention is expressed by the recombinant Listeria strain. In another embodiment, the expression is mediated by a nucleotide molecule carried by the recombinant Listeria strain. Each possibility represents a separate embodiment of the present invention.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. Each possibility represents a separate embodiment of the present invention.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art. Each possibility represents a separate embodiment of the present invention.

In another embodiment, a vaccine of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art. Each possibility represents a separate embodiment of the present invention.

In one embodiment, the method provided herein further comprises the step of co-administering with, prior to or following the administration of said recombinant Listeria strain an immunogenic composition comprising an immune checkpoint protein inhibitor. In another embodiment, the immunogenic composition is the immune checkpoint protein inhibitor. It will be appreciated by the skilled artisan that any immune checkpoin protein known in the art can be targeted by an immune check point inhibitor. An immune checkpoint protein may be selected from, but is not limited to the following: programmed cell death protein 1 (PD1), T cell membrane protein 3 (TIM3), adenosine A2a receptor (A2aR) and lymphocyte activation gene 3 (LAGS), killer immunoglobulin receptor (KIR) or cytotoxic T-lymphocyte antigen-4 (CTLA-4). In another embodiment, the checkpoint inhibitor protein is one belonging to the B7/CD28 receptor superfamily.

In one embodiment, the methods provided herein further comprise the step of co-administering an immunogenic composition comprising a cytokine that enhances an anti-tumor immune response in said subject. Cytokines that serve to enhance an immune response are well known and will be appreciated by the skilled artisan to include, type I interferons (IFN-α/IFN-β), TNF-α, IL-1, IL-4, IL-12, INF-γ, and any other cytokine known to enhance an immune response. In another embodiment, the cytokine is an inflammatory cytokine.

It will be well appreciated an “immunogenic composition” may comprise the recombinant listeria provided herein, and an adjuvant, an immune checkpoint protein inhibitor, and a cytokine provided herein. In another embodiment, an immunogenic composition comprises a recombinant Listeria provided herein. In another embodiment, an immunogenic composition comprises an adjuvant known in the art or as provided herein. In another embodiment, an immunogenic composition comprises an immune checkpoint inhibitor known in the art or as provided herein. In another embodiment, an immunogenic composition comprises cytokine known in the art or as provided herein. It is also to be understood that such compositions enhance an immune response, or increase a T effector cell to regulatory T cell ratio or elicit an anti-tumor immune response, as further provided herein.

Following the administration of the recombinant listeria provided herein, alone or when co-administered with the immunogenic compositions provided herein, the methods provided herein induce the expansion of T effector cells in peripheral lymphoid organs leading to an enhanced presence of T effector cells at the tumor site. In another embodiment, the methods provided herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs, as demonstrated herein (see Examples). It will be appreciated by the skilled artisan that peripheral lymphoid organs include, but are not limited to, the spleen, payer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites.

In one embodiment, combining the attenuated recombinant Listeria strains that express a fusion protein of truncated LLO and a heterologous antigen with a recombinant Listeria expressing the same antigen leads to complete tumor regression as demonstrated herein (see Example 6).

In another embodiment, a recombinant nucleic acid of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto.

### Pharmaceutical Compositions

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In one embodiment, a composition of this invention comprises a recombinant Listeria monocytogenes (Lm) strain.

As used throughout, the terms “composition”, “vaccine” and “immunogenic composition” are interchangeable having all the same meanings and qualities. The term “pharmaceutical composition” refers, in some embodiments, to a composition suitable for pharmaceutical use, for example, to administer to a subject in need. In another embodiment, the term “pharmaceutical composition” encompasses a therapeutically effective amount of the active ingredient or ingredients including the Listeria strain, together with a pharmaceutically acceptable carrier or diluent. It is to be understood that the term a “therapeutically effective amount” refers to that amount which provides a therapeutic effect for a given condition and administration regimen.

Compositions of this invention may be used in methods of this invention in order to elicit an enhanced anti-tumor T cell response in a subject, in order to inhibit tumor-medicated immunosuppression in a subject, or for increasing the ratio or T effector cells to regulatory T cells (Tregs) in the spleen and tumor of a subject, or any combination thereof.

In another embodiment of the methods and compositions provided herein, a composition is administered orally, and is thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, a composition provided herein is administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

It will be understood by the skilled artisan that the term “administering” encompasses bringing a subject in contact with a composition of the present invention. In one embodiment, administration can be accomplished in vitro, i.e. in a test tube, or in vivo, i.e. in cells or tissues of living organisms, for example humans. In one embodiment, the present invention encompasses administering the Listeria strains and compositions thereof of the present invention to a subject.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the singular form “a,” “an” and “the” include plural references unless the context clearly dictates otherwise. For example, the term “a compound” or “at least one compound” may include a plurality of compounds, including mixtures thereof.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

Mice

C57BL/6 mice, female, 6-8-week-old (unless stated), were purchased from Frederick National Laboratory for Cancer Research (FNLCR). Mice were housed in the Animal Facility of National Cancer Institute, Bethesda. Protocols for use of experimental mice were approved by the Animal Care and Use Committee at National Institutes of Health.

Cell Line

TC-1 cells, which express low levels of E6 and E7, was derived from primary C57BL/6 mice lung epithelial cells by transformation with HPV-16 E6 and E7 and activated ras oncogene. The cells were grown in RPMI 1640, supplemented with 10% FBS, 100 U/ml penicillin, 100 μg/ml streptomycin, 2 mM L-glutamine, 1 mM sodium pyruvate, 100 μM nonessential amino acids, and 0.4 mg/ml G418 at 37° C. with 5% CO₂.

L. monocytogenes Strains

LmddA-LLO-E7 and its controls LmddA-LLO and LmddA were generated in Advaxis Inc (Princeton, N.J.). The dal dat AactA strain (LmddA) was constructed from the dal dat strain, which is based on Lm wild-type strain 10403S with a streptomycin resistance gene integrated into the chromosome. With dal, dat, and actA mutated, LmddA is highly attenuated. LmddA-LLO-E7 strain was constructed by transformation of LmddA with pTV3 plasmid after deletion of prfA, as well as the chloramphenicol resistance gene in the plasmid. Expression and secretion of LLO-E7 fusion protein was confirmed in the culture supernatants of LmddA-LLO-E7 strain by Western blotting as previously described. Construction of LmddA-LLO control strain was similar as that of LmddA-LLO-E7 strain but both prfA and E7 were deleted in pTV3 plasmid. Lm wild-type strain 10403S and some mutant strains, including Δhly, Δhly::pfo, and hly::Tn917-lac (pAM401-hly) were kindly provided by Dr. D. Portnoy (University of California, Berkeley, Calif.). The strain hly::Tn917-lac is a nonhemolytic mutant of wild-type Lm, in which the Tn917-lac fusion gene is inserted into the hly gene (the gene encoding LLO) to disrupt LLO hemolytic activity. When this mutant is transfected with a plasmid that expresses LLO (pAM401-hly), it gains hemolytic activity again since it has LLO. Lm-E7 strain, in which the full length of E7 gene was integrated into Lm chromosome, was kindly provided by Dr. Y. Paterson (University of Pennsylvania, Philadelphia, Pa.). Bacteria were cultured in brain heart infusion medium plus streptomycin (100 μg/ml) and in presence or absence of D-alanine (100 μg/ml).

Reagents

Fluorescence conjugated anti-mouse antibodies CD4-PerCP-Cy5.5 (GK1.5) and CD8-Brillient Violet 421 (53-6.7) were from Biolegend (San Diego, Calif.). FoxP3-FITC (FJK-16s) was from eBioscience (San Diego, Calif.). H-2D^{b} tetramers loaded with the E7 peptide (RAHYNIVTF) SEQ ID NO: 5 was kindly provided by the National Institute of Allergy and Infectious Diseases Tetramer Core Facility and the National Institutes of Health AIDS Research and Reference Reagent Program. CountBright™ absolute counting beads were from Life Technologies (Grand Island, N.Y.).

Tumor Inoculation and Mice Vaccination

TC-1 cells (10⁵ cells/mouse) were implanted s.c. in the right flank of mice on day 0. On day 10, when tumor became 5-6 mm in diameter, mice were injected i.p. with LmddA-LLO-E7 vaccine or proper controls at a dose of 0.1 LD50. Vaccination was boosted on day 17. Tumor was measured twice a week using an electronic caliper and tumor size was calculated by the formula: length×width×width/2. Mice were euthanized when tumor reached 2.0 cm in diameter.

Flow Cytometry

Mouse splenocytes or cells harvested from tumor were stained with CD4-PerCP-Cy5.5, CD8-Brillient Violet 421, and H-2D^{b} E7 tetramer-APC for 30 min Cells were fixed, permeabilized, and stained with FoxP3-FITC overnight. Cells were analyzed by flow cytometry. A lymphocyte gate was set where Tregs were identified as CD4+FoxP3+. CountBright™ absolute counting beads were added for counting absolute cell numbers.

Adoptive Transfer of CD4+CD25+ Tregs

CD4+CD25+ T cells were isolated from mouse spleens by Dynal® CD4+CD25+ Treg Kit (Life Technologies, Grand Island, N.Y.). Cells were injected i.v. into TC-1 tumor-bearing mice at day 9 post tumor cell inoculation. One day after Treg transfer, mice were immunized i.p. with LmddA-LLO-E7 (0.1 LD50) twice at one week interval. Tumor growth was monitored.

Statistics

The data were analyzed using the nonparametric Mann-Whitney test. Significance was determined at P<0.05.

### Example 1

### LmddA-LLO-E7 Induces Regression of Established TC-1 Tumors Accompanied by Treg Frequency Decrease

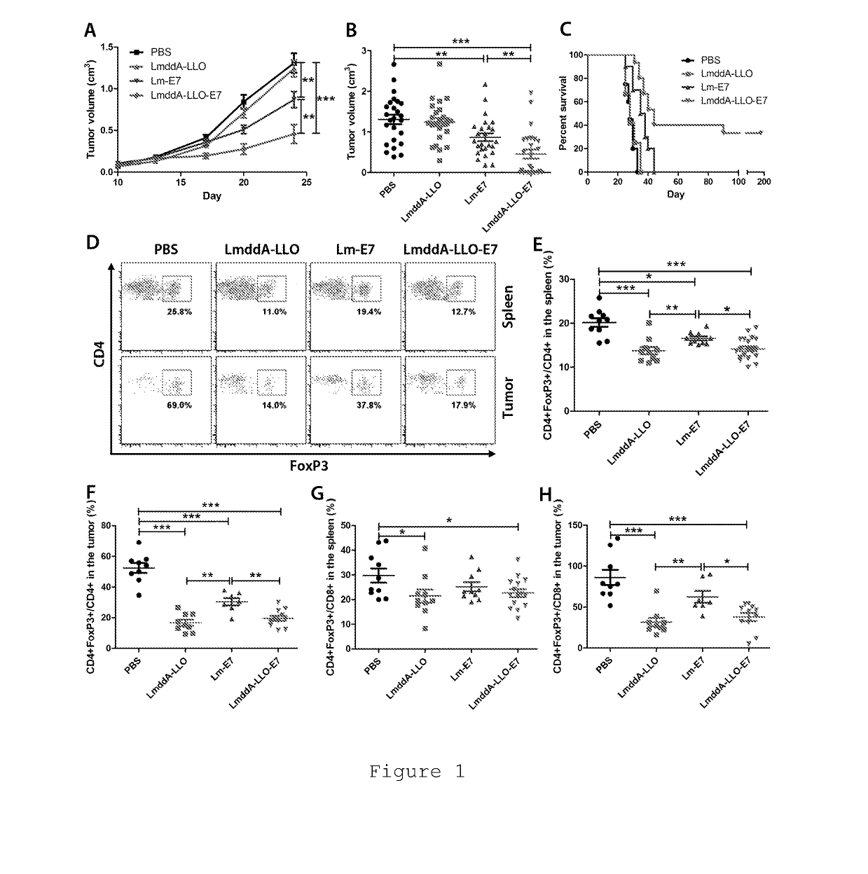

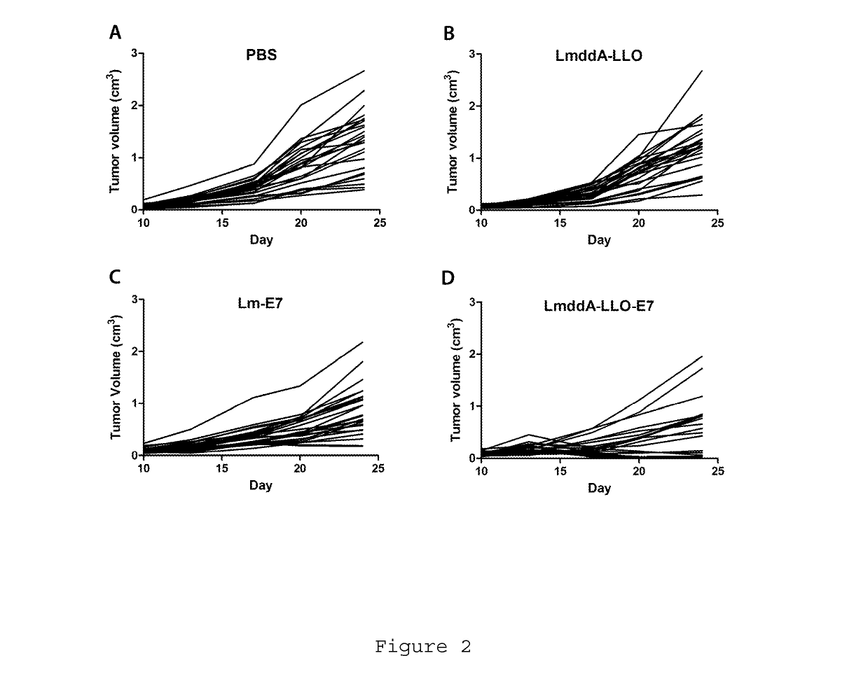

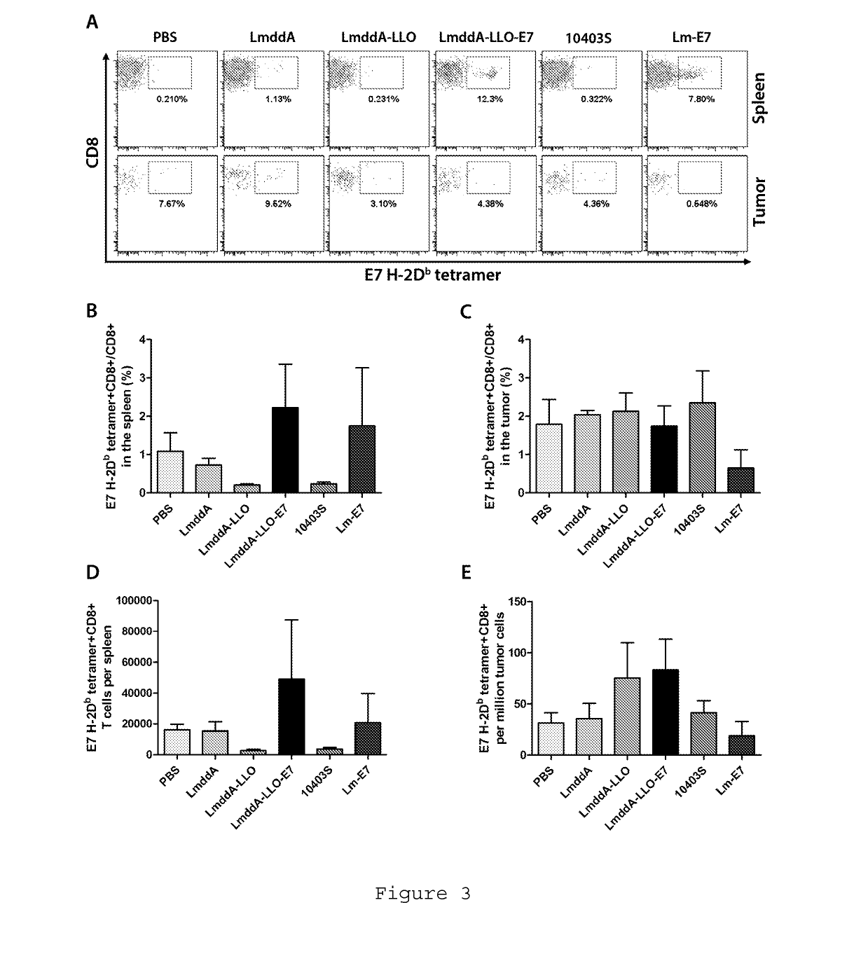

It was previously reported that a Lm-based vaccine, Lm-LLO-E7, where a fusion protein LLO-E7, as well as PrfA, is expressed episomally in a prfA negative strain of Listeria XFL-7, induced complete regression of established TC-1 tumors. Here the antitumor activity of another highly attenuated Lm-based vaccine, LmddA-LLO-E7, which produces the fusion protein LLO-E7 by a plasmid in a dal, dat, and actA mutated Lm strain, was investigated. LmddA-LLO-E7 is more attenuated compared to Lm-LLO-E7, since the chloramphenicol resistance gene and PrfA have been removed from the plasmid. It was observed that similar to Lm-LLO-E7, LmddA-LLO-E7 significantly inhibited the growth of established TC-1 tumors (FIGS. 1, A and B, FIG. 2). Tumor completely regressed in approximately 40% of TC-1 tumor-bearing mice after vaccination with LmddA-LLO-E7 twice (FIG. 1B and FIG. 2). Except for one mouse that relapsed and died at 3 months, the others that showed tumor regression (33% of total animals) survived at least 6 months without relapse (FIG. 1C). Although Lm-E7 slowed down TC-1 tumor growth, it failed to induce complete tumor regression (FIGS. 1, A and B and FIG. 2). LmddA-LLO (without E7) was unable to significantly inhibit TC-1 tumor growth (FIGS. 1, A and B and FIG. 2), suggesting that innate immune response is not sufficient to eradicate TC-1 tumor cells. LmddA-LLO-E7 and Lm-E7 induced similar H-2D^{b} E7 tetramer+CD8+ T cell response in the spleen (FIGS. 3, A-upper panel, B, and D), which was consistent with previous finding. CD4+FoxP3+ Tregs were then analyzed. Unexpectedly, it was observed that LmddA-LLO-E7, Lm-E7, and LmddA-LLO, all significantly decreased Treg frequency in the spleen and more dramatically in the tumor compared to PBS control, though LmddA-LLO-E7 and LmddA-LLO decreased the frequency more than did Lm-E7 (FIG. 1, D-H).

### Example 2

### Lm is Sufficient to Induce Decrease of Treg Frequency

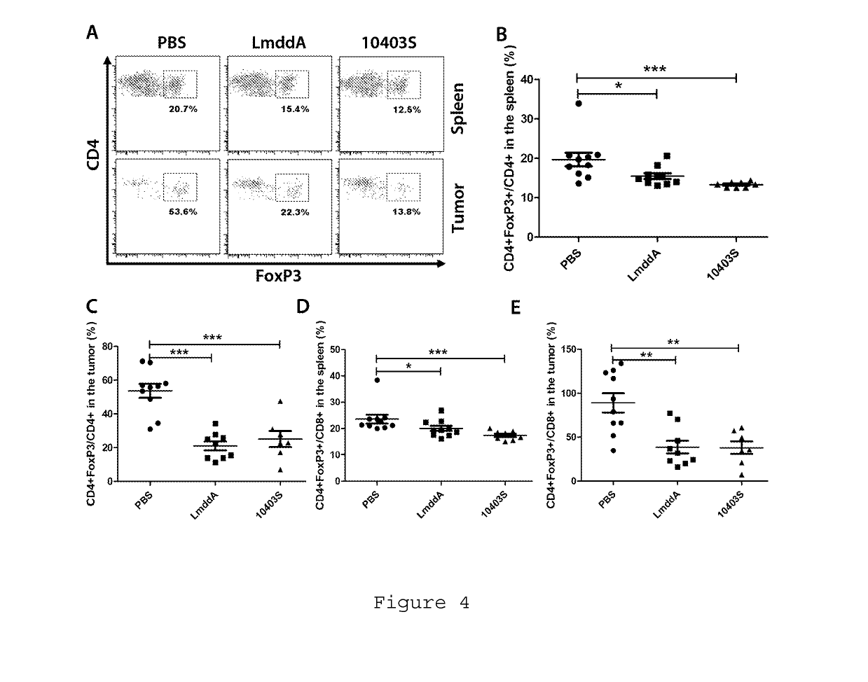

Initially, it was suspected that the decrease of Treg frequency was mediated by the truncated LLO. But Lm-E7, without expression of the truncated LLO, was also able to decrease Treg frequency (FIG. 1, D-H). This observation suggests that Lm might be able to decrease Treg frequency. Indeed, both LmddA, the vector control for LmddA-LLO-E7, and 10403S, a wild-type Lm strain and the vector control for Lm-E7, significantly decreased Treg frequency in the spleen and more so in the tumor (FIG. 4).

### Example 3

### Lm Decreases Treg Frequency by Preferentially Inducing CD4+FoxP3− T Cell and CD8+ T Cell Expansion

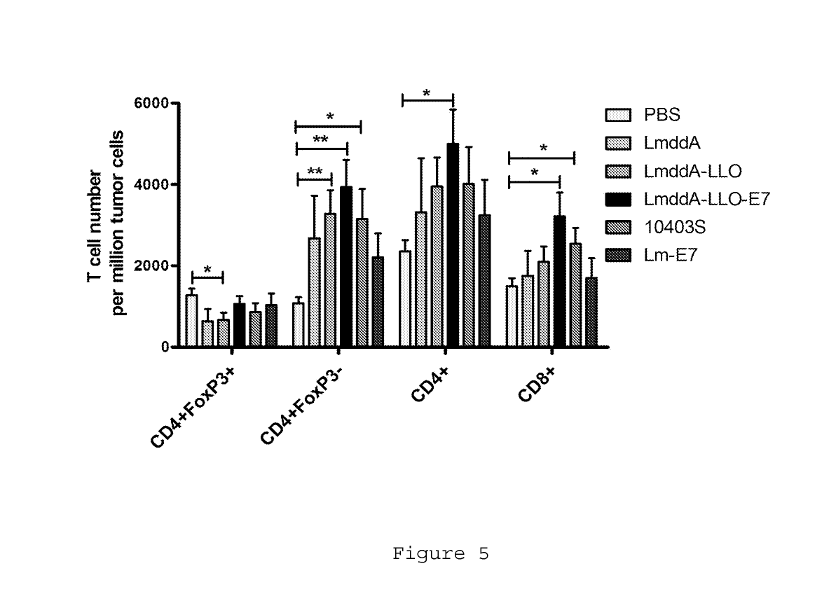

A relative Treg frequency (proportion of total T cells) is determined not only by the number of Tregs but also by the number of CD4+FoxP3− T cells and CD8+ T cells. To investigate how Lm decreases Treg frequency, CD4+FoxP3+ Treg, CD4+FoxP3− T cell and CD8+ T cell number were quantified in TC-1 tumor-bearing mice treated with LmddA-LLO-E7, LmddA-LLO, LmddA, Lm-E7, or Lm (10403S). As shown in FIG. 5, surprisingly, it was found that LmddA did not markedly change the number of CD4+FoxP3+ T cells in the tumor. It actually increased CD4+FoxP3− T cells and CD8+ T cells, thus decreasing Treg frequency proportionately. Episomal expression of a truncated LLO in LmddA-LLO and LmddA-LLO-E7 further increased CD4+FoxP3− T cells and CD8+ T cells, thus decreasing CD4+FoxP3+ T cell frequency more. Wild-type Lm 10403S and Lm-E7 also induced an increase in CD4+FoxP3− T cells and CD8+ T cells while not significantly changing CD4+FoxP3+ T cell number. Lm-LLO-E7 significantly increased the density of CD4+FoxP3− T cells and CD8+ T cells in the tumor. These results demonstrate that Lm preferentially induces CD4+FoxP3− T cell and CD8+ T cell expansion to decrease CD4+FoxP3+ T cell frequency.

### Example 4

### Lm-Induced Expansion of CD4+FoxP3− T Cells and CD8+ T Cells is Dependent on and Mediated by LLO

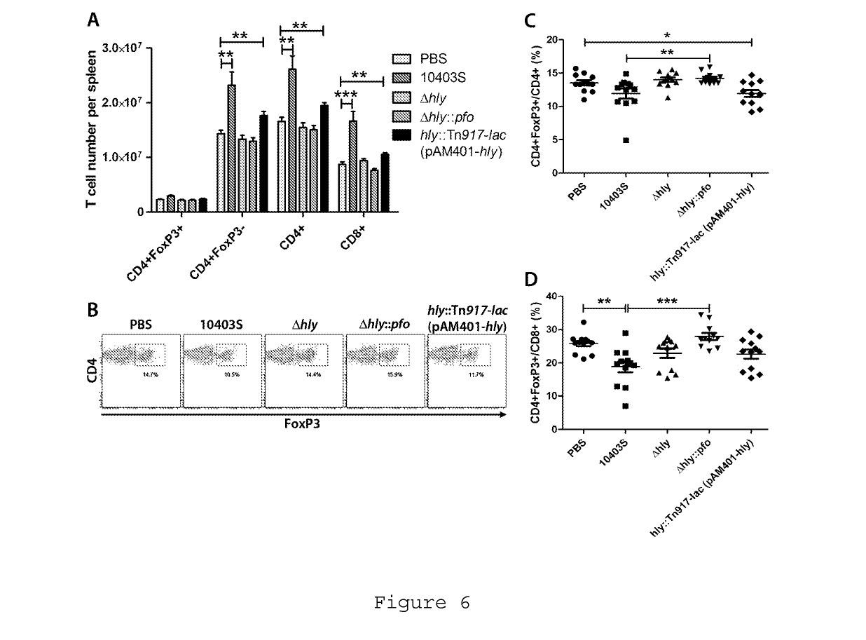

LLO, encoded by the hly gene, is a pore-forming cytolysin by which Lm can escape from a host cell phagosomal vacuole into the cytoplasm. Since LmddA-LLO-E7, Lm-E7 and all their controls produce LLO, a LLO-deficient Lm mutant derived from 10403S, in which hly gene is deleted using a shuttle vector followed by homologous recombination, was used to study if LLO plays a role in inducing expansion of CD4+FoxP3− T cells and CD8+ T cells. It was found that Δhly Lm was unable to increase CD4+FoxP3− T cells and CD8+ T cells in the spleen of mice on day 7 after a single administration (FIG. 6A), indicating that induction of CD4+FoxP3− T cell and CD8+ T cell expansion is dependent on LLO. This could be a direct effect of LLO or a requirement to escape the phagolysosome. To address this question, we studied an LM with LLO replaced by PFO. Perfringolysin O (PFO), produced by Clostridium perfringens, is 43% identical in amino acids with LLO and can also lyse the vacuolar membrane. The pfo gene, encoding PFO under the control of hly promoter, was recombined into the chromosome of the Δhly strain to form Δhly::pfo strain. Although Δhly::pfo was able to escape from phagocytosis into the cytoplasm, it was unable to increase CD4+FoxP3− T cells and CD8+ T cells in the mouse spleen (FIG. 6A). In contrast, hly::Tn917-lac (pAM401-hly), a nonhemolytic Tn917-lac mutant of wild-type Lm (in which Tn917-lac fusion gene is inserted into the hly gene to disrupt LLO hemolytic activity) transformed with a LLO expressing plasmid pAM401-hly, induced expansion of mouse splenic CD4+FoxP3− T cells and CD8+ T cells (FIG. 6A). These results suggest that expansion of CD4+FoxP3− T cells and CD8+ T cells is directly mediated by LLO. Since Lm did not induce CD4+FoxP3+ T cell expansion significantly, Lm-induced Treg decrease in frequency resulted from the increase of CD4+FoxP3− T cells and CD8+ T cells (FIG. 6, A-D).

### Example 5

### Episomal Expression of a Truncated LLO in LmddA Induces Expansion of CD4+FoxP3− T Cells and CD8+ T Cells to a Higher Level

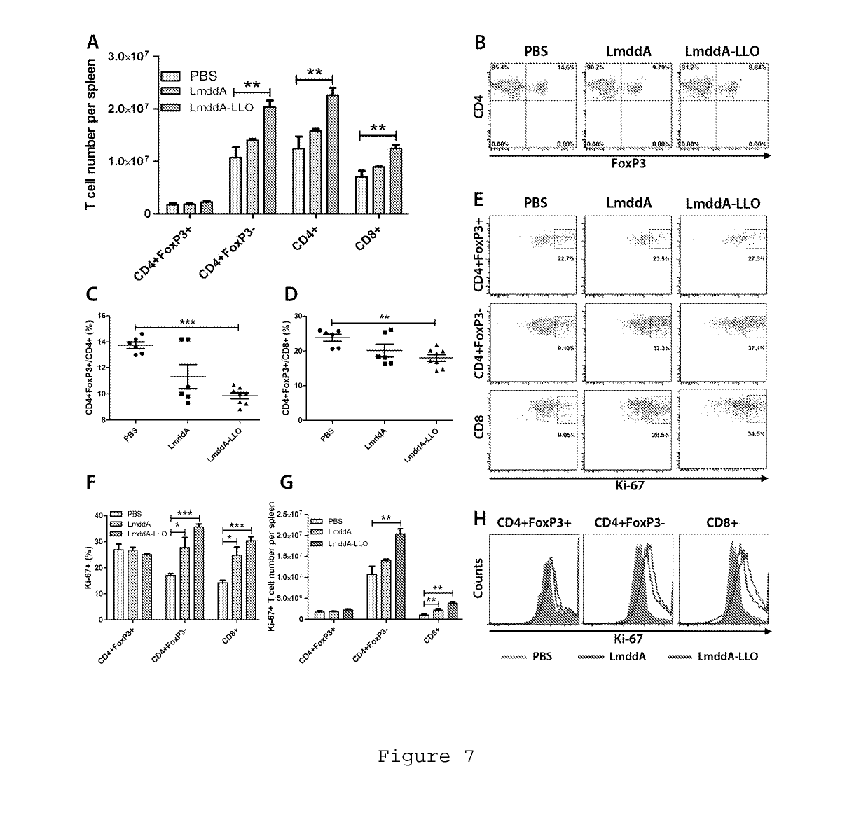

Next LmddA and LmddA-LLO were compared, in which the latter produces a truncated LLO episomally by a plasmid, in induction of T cell proliferation in healthy, non-tumor-bearing mice. It was found that LmddA was able to slightly increase CD4+FoxP3− T cell and CD8+ T cell number in the spleen of mice at day 7 after a single administration, but LmddA-LLO further induced such an increase to a higher level (FIG. 7A). In contrast, CD4+FoxP3+ T cell number was not significantly changed after LmddA or LmddA-LLO infection (FIG. 7A). These resulted in a significant decrease of Tregs in proportion after LmddA-LLO administration compared to PBS control (FIG. 7, B-D). The presence of the cell proliferation marker Ki-67 in these cells, was examined. LmddA increased the frequency and absolute number of Ki-67+CD4+FoxP3− T cells and Ki-67+CD8+ T cells, but LmddA-LLO increased the number to a greater extent (FIG. 7, E-G). The level of Ki-67 expression in CD4+FoxP3− T cell and CD8+ T cells was also increased accordingly (FIG. 7H). In contrast, the frequency and absolute number of Ki-67+CD4+FoxP3+ T cells and Ki-67 expression in CD4+FoxP3+ T cells was not markedly changed, indicating LmddA and LmddA-LLO did not induce their proliferation.

### Example 6

### The Combination of Lm-E7 and LmddA-LLO Induces Regression of Established TC-1 Tumors

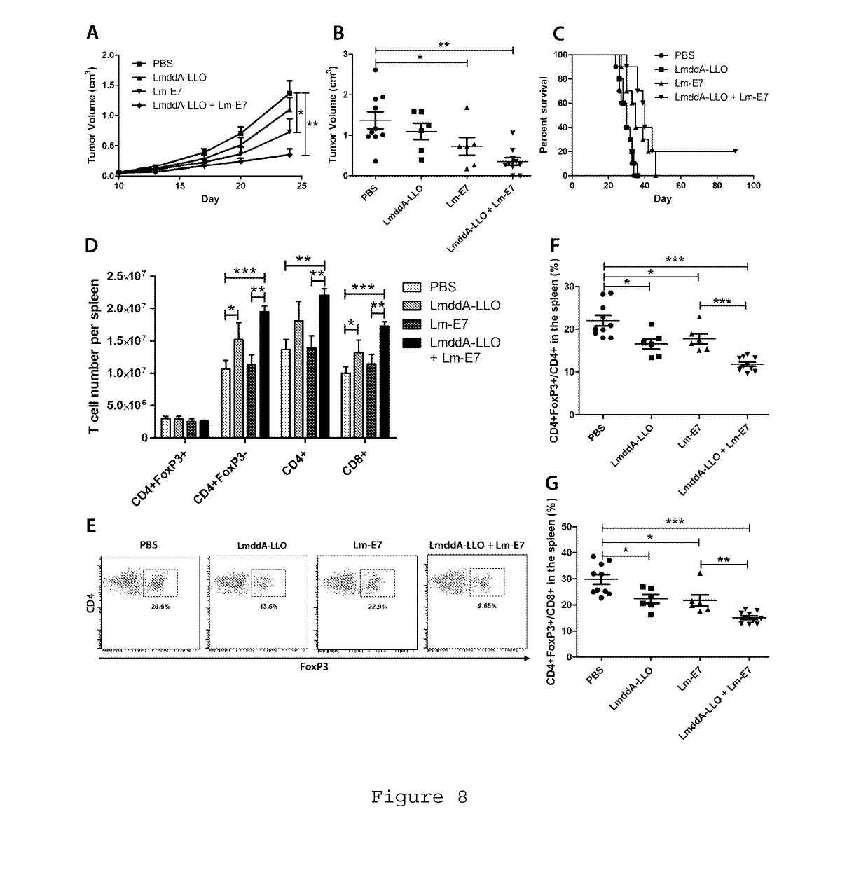

The Lm-E7 vaccine alone did not induce much expansion of CD4+FoxP3− T cells and CD8+ T cells (FIG. 5). This may account for its failure in induction of TC-1 tumor regression. Since LmddA-LLO induced CD4+FoxP3− T cell and CD8+ T cell expansion (FIG. 5 and FIG. 7A), it is conceivable that the anti-tumor effect of Lm-E7 may be improved in the presence of LmddA-LLO. Indeed, the combination of Lm-E7 and LmddA-LLO induced nearly complete regression of established TC-1 tumors (FIG. 8, A-C). In contrast, addition of LmddA failed to augment Lm-E7-induced anti-tumor activity (Data not shown), indicating the importance of the truncated non-hemolytic LLO in improving the anti-tumor efficacy of Lm-E7 vaccine. As expected, CD4+FoxP3− T cell and CD8+ T cell number was significantly increased in the spleen of the combination group mice compared with those treated with Lm-E7 or PBS (FIG. 8D). Again, because CD4+FoxP3+ number was relatively unchanged, the increase of CD4+FoxP3− T cell and CD8+ T cell number to a higher level by combined Lm-E7 and LmddA-LLO resulted in a greater decrease in the CD4+FoxP3+ T cell proportion (FIG. 8, E-G).

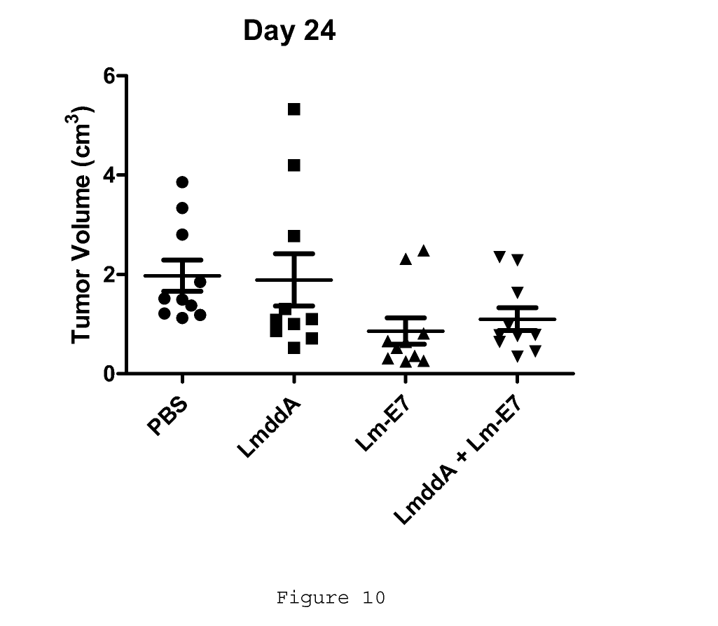

Moreover, LmddA was also co-administered with Lm-E7 as a control to determine the role the non-hemolytic truncated LLO played during co-administration of LmddA-LLO and Lm-E7. It was observed that the addition of the LmddA strain failed to augment the Lm-E7 induced anti-tumor activity, indicates that the endogenous LLO produced by LmddA could not assist Lm-E7-induced anti-tumor activity (FIG. 10).

### Example 7

### Adoptive Transfer of Tregs Compromises the Anti-Tumor Efficacy of LmddA-LLO-E7 Against Established TC-1 Tumors

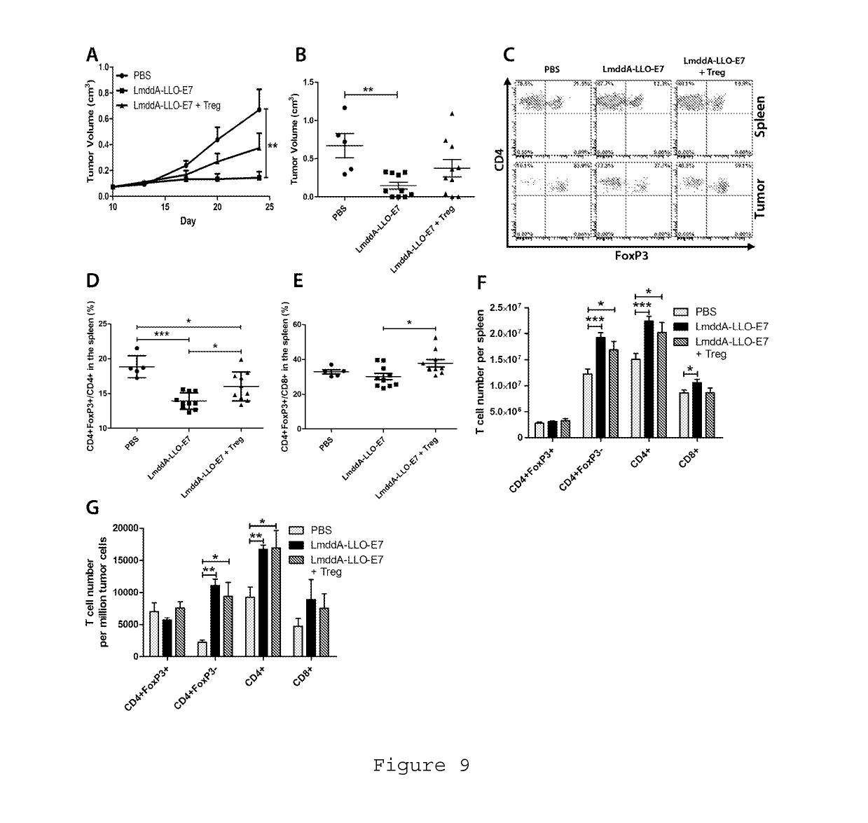

LmddA-LLO-E7 did not significantly change Treg numbers, although it decreased Treg frequency (FIG. 1, D-H). The ratio of Tregs to CD4+FoxP3− T cells or to CD8+ T cells has been a well-accepted parameter to determine Treg suppressive ability. To determine whether the Treg proportion has any impact on the anti-tumor efficacy of LmddA-LLO-E7, we isolated CD4+CD25+ Tregs from naïve C57BL/6 mice and injected them i.v. into TC-1 tumor-bearing mice, which were followed by LmddA-LLO-E7 vaccination. LmddA-LLO-E7 significantly inhibited TC-1 tumor growth in the mice without adoptive transfer of Tregs (FIGS. 9, A and B). However, in the mice given Tregs, LmddA-LLO-E7 was unable to significantly inhibit TC-1 tumor growth (FIGS. 9, A and B). Mice receiving Tregs showed a slight increase of Treg number in the spleen but more decrease in the tumor. On the other hand, mice receiving Tregs had fewer CD4+FoxP3− T cells and CD8+ T cells after being vaccinated with LmddA-LLO-E7 compared to the LmddA-LLO-E7 control, indicating adoptive transfer of Tregs inhibits CD4+FoxP3− T cell and CD8+ T cell expansion (FIGS. 9, F and G). These together resulted in the increase of Treg frequency in the Treg-recipient mice (FIG. 9, C-E).

Tumor antigen-specific CTLs play dominant roles in killing tumor cells, and Lm, as an intracellular bacteria, can deliver antigens associated with MHC class I molecules to activate CTLs. However, why did two Lm-based vaccines, Lm-LLO-E7 and Lm-E7, induce similar levels of HPV E7-specific CTLs in the spleen but nevertheless exhibit distinct anti-tumor activity, with the former (Lm-LLO-E7) inducing a much stronger anti-tumor effect? (FIG. 1, A-C, FIG. 2, FIG. 3). It is no doubt that CD8+ T cells participate in killing tumor cells, as their depletion abrogated Lm-LLO-E7-induced tumor regression. It is also clear that a certain level of tumor-antigen specific CTLs is necessary for killing tumor cells, as LmddA-LLO, which lacks E7 expression, was unable to significantly inhibit TC-1 tumor growth (FIG. 1, A-C and FIG. 2). It has been proposed that Lm-E7 induced an increase of Tregs to suppress the host immune response, thus compromising its anti-tumor immunity. However, in our hands, we found that actually both Lm-E7 and LmddA-LLO-E7 decreased Treg frequency in a TC-1 tumor model compared to PBS control (FIG. 1, D-H). Furthermore, it was found that neither Lm-E7 nor LmddA-LLO-E7 significantly increased Treg total number in TC-1 tumor after vaccination (FIG. 5).

In fact, it was found that a major difference between LmddA-LLO-E7 and Lm-E7 is that the former was able to induce a marked increase of CD4+FoxP3− T cell and CD8+ T cell number while the latter induced an increase to a much less degree (FIG. 5). This explains why LmddA-LLO-E7 decreased Treg percentage to a greater degree than Lm-E7 (FIG. 1, D-H). It was observed that Lm vector was sufficient to increase CD4+FoxP3− T cell and CD8+ T cell number. However, with episomal expression of a truncated LLO, Lm increased CD4+FoxP3− T cell and CD8+ T cell number dramatically to a higher level, thus decreasing Treg frequency even further (FIG. 7). Thus, it is conceivable that LLO plays a critical role in inducing increase of CD4+FoxP3− T cell and CD8+ T cell number. Indeed, LLO is not only necessary for L. monocytogenes to escape from the phagosome but also directly causes CD4+FoxP3− T cell and CD8+ T cell expansion, as neither a LLO-minus (Δhly) L. monocytogenes strain nor a Δhly::pfo strain, which expresses PFO that enables Lm to enter the cytoplasm, succeeded in inducing CD4+FoxP3− T cell and CD8+ T cell proliferation, but transformation of a nonhemolytic LLO mutant Lm strain with an LLO-expressing plasmid restored CD4+FoxP3− T cell and CD8+ T cell expansion (FIG. 6). LLO-induced CD4+FoxP3-T cell and CD8+ T cell expansion is unrelated to its hemolytic activity, as episomal expression of a nonhemolytic truncated LLO in LmddA greatly augmented CD4+FoxP3− T cell and CD8+ T cell expansion (FIG. 7). Although the expansion of both CD4+ T cell and CD8+ T cell responses by LLO appears to be an antigen-non-specific adjuvant effect, LLO may also contain immuno-dominant epitopes of these two cell types. Indeed, early studies identified that LLO bears two CD4+ T cell epitopes (residues 189-201 and residues 215-226, respectively) and one CD8+ T cell epitope (residues 91-99).

LmddA-LLO-E7's excellent anti-tumor effect is likely due to the fact that it induces a significant increase of CD4+FoxP3− T cells and CD8+ T cells. In contrast, the inability of Lm-E7 to induce marked increase of CD4+FoxP3− T cell and CD8+ T cell number accounts for its inefficiency in eradication of tumors, as the combination of Lm-E7 and LmddA-LLO, which dramatically increased CD4+FoxP3− T cell and CD8+ T cell number compared to Lm-E7 alone, induced nearly complete regression of established TC-1 tumors (FIG. 8). Our data indicate that the LmddA-LLO-E7-induced decrease in Treg frequency is the consequence of an increase in CD4+FoxP3− T cell and CD8+ T cell number. The ratio of Tregs to CD4+FoxP3− T cells or to CD8+ T cells is critical, to suppress the function of CD4+FoxP3− T cells and CD8+ T cells. Indeed, increasing the Treg ratio in vivo by adoptive transfer of Tregs to tumor-bearing mice followed by LmddA-LLO-E7 vaccination inhibited the expansion of CD4+FoxP3− T cells and CD8+ T cells and consequently compromised the vaccine's anti-tumor efficacy (FIG. 9).

Besides preferentially inducing the expansion of CD4+FoxP3− T cells and CD8+ T cells, the truncated non-hemolytic LLO makes other contributions to improving the anti-tumor efficacy of LmddA-LLO-E7 vaccine. It was observed that although Lm-E7 and LmddA-LLO-E7 induced similar expansion of E7-specific CD8+ T cells, but this is not the case in the tumor. With episomal expression of the truncated LLO (LmddA-LLO-E7), more E7-specific CD8+ T cells tended to be induced in the tumor (FIG. 3E). We found that LmddA-LLO-E7 upregulated the expression of chemokine receptors CCR5 and CXCR3 on CD4+FoxP3− T cells and CD8+ T cells, but not on CD4+FoxP3+ T cells showing that CCR5 and CXCR3 are crucial for Th1 and CD8+ T cell trafficking. These results suggest that LLO induces CD4+FoxP3− T cell and CD8+ T antigen-specific cell migration to the tumor micronvironment through upregulation of CCR5 and CXCR3. In addition, it is known that truncated LLO is required for the efficient secretion of the antigen from Lm, and antigens that are not secreted from the Lm vector result in the induction of less effective anti-tumor immunity. Hence, the lack of potent anti-tumor activity of the Lm-E7 vector might not only be due to the lack of effectively expanding the CD4+FoxP3− T cells and CD8+ T cells but also be due to the inefficient secretion of the antigen from Lm in context of an infected antigen presenting cell and the priming of an ineffective antigen-specific T cell response.

Overall, it was demonstrated that episomal expression of a nonhemolytic truncated LLO in a LmddA-LLO-E7 vaccine preferentially induces CD4+FoxP3− T cell and CD8+ T cell expansion, which enhances the vaccine's anti-tumor activity. In conclusion, the results show that many factors, like a certain level of antigen-specific CTLs, and of non-tumor antigen-specific CD4+FoxP3− T cells and CD8+ T cells, and a decreased Treg proportion, are all needed to trigger an effective anti-tumor immune response, and that this can be accomplished with the Listeria constructs provided herein. Further, this work indicates that LLO is a promising vaccine adjuvant in that it preferentially induces CD4+FoxP3− T cell and CD8+ T cell expansion, thus overall decreasing Treg frequency and favoring immune responses to kill tumor cells.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of eliciting an anti-tumor T cell response in a subject having a tumor or cancer, comprising the step of administering to said subject a recombinant Listeria strain comprising a recombinant nucleic acid, said nucleic acid molecule comprising a first open reading frame encoding a recombinant polypeptide and a second open reading frame second open reading frame encoding a metabolic, wherein said recombinant polypeptide comprises a truncated LLO protein fused to a heterologous antigen or fragment thereof, wherein said Listeria comprises a mutation in the endogenous alanine racemase gene (dal), D-amino acid transferase gene (dat), and actA genes, and wherein said T-cell response comprises increasing a ratio of T effector cells to regulatory T cells (Tregs), thereby eliciting an anti-tumor T cell response in said subject.

2. The method of claim 1, wherein said tumor-associated antigen is a human papilloma virus E7 antigen.

3. The method of any one of claims 1-2, wherein said truncated LLO protein is an N-terminal LLO.

4. The method of claim 2, wherein said LLO is set forth in SEQ ID NO: 2.

5. The method of any one of claims 1-4, wherein said heterologous antigen is a tumor-associated antigen.

6. The method of any one of claims 1-4, wherein said tumor-associated antigen is an angiogenic antigen.

7. The method of any one of claims 1-6, wherein said Listeria lacks antibiotic resistance genes.

8. The method of any one of claims 1-7, wherein said recombinant nucleic acid is in a plasmid in said Listeria.

9. The method of claim 8, wherein said plasmid is an episomal plasmid.

10. The method of claim 9, wherein said plasmid is a multicopy plasmid.

11. The method of claim 8, wherein said plasmid is an integrative plasmid.

12. The method of any one of claims 1-11, wherein said metabolic enzyme is an amino acid metabolism enzyme.

13. The method of claim 12, wherein said metabolic enzyme is a D-amino acid transferase enzyme or a alanine racemase enzyme.

14. The method of any one of claims 1-13, further comprising administering to said subject an adjuvant.

15. The method of claim 14, wherein said adjuvant comprises a granulocyte/macrophage colony-stimulating factor (GM-CSF), a saponin QS21, a monophosphoryl lipid Aa CpG-containing oligonucleotide, or a bacterial toxin.

16. The method of any one of claims 1-15, further comprising co-administering with, prior to or following the administration of said recombinant Listeria an immune checkpoint protein inhibitor.

17. The method of claim 16, wherein said immune checkpoint protein is programmed cell death protein 1 (PD1), T cell membrane protein 3 (TIM3), adenosine A2a receptor (A2aR) and lymphocyte activation gene 3 (LAGS), killer immunoglobulin receptor (KIR) or cytotoxic T-lymphocyte antigen-4 (CTLA-4).

18. The method of any one of claim 1-15, or 1-16, further comprising co-administering a cytokine that enhances said anti-tumor immune response.

19. The method of claim 18, wherein said cytokine is: a type I interferon (IFN-α/IFN-(3), TNF-α, IL-1, IL-4, IL-12, INF-γ.

20. The method of any one of claims 1-19, wherein said method induces the expansion of T effector cells in peripheral lymphoid organs leading to an enhanced presence of T effector cells at the tumor site.

21. The method of claim 20, wherein said expansion of T effector cells leads to an increased ratio of T effector cells to regulatory T cells in the periphery and at the tumor site without affecting the number of Tregs.

22. The method of claim 21, wherein said T effector cells are CD4+FoxP3- and CD8+ T-cells.

23. The method of claim 21, wherein said T effector cells are CD4+FoxP3− T cells.

24. The method of claim 21, wherein said regulatory T cells are CD4+FoxP+ T cells.

25. A method for increasing the ratio of T effector cells to regulatory T cells (Tregs) in the spleen of a subject, the method comprising the step of administering to said subject a recombinant Listeria strain comprising a recombinant nucleic acid encoding a truncated LLO protein, wherein said Listeria comprises a mutation in the endogenous alanine racemase gene (dal), D-amino acid transferase gene (dat), and actA genes, wherein said T-cell response comprises increasing a ratio of T effector cells to regulatory T cells (Tregs).

26. The method of claim 25, wherein said tumor-associated antigen is a human papilloma virus E7 antigen.

27. The method of any one of claims 25-26, wherein said truncated LLO protein is an N-terminal LLO.

28. The method of any one of claims 25-27, wherein said LLO is set forth in SEQ ID NO: 2.

29. The method of any one of claims 25-28, wherein said Listeria lacks an antibiotic resistance genes.

30. The method of any one of claims 25-29, wherein said nucleic acid is in a plasmid in said Listeria.

31. The method of claim 30, wherein said plasmid is an episomal plasmid.

32. The method of claim 31, wherein said plasmid is a multicopy plasmid.

33. The method of claim 30, wherein said plasmid is an integrative plasmid.

34. The method of any one of claims 25-33, further comprising administering to said subject an adjuvant.

35. The method of any one of claims 25-34, wherein said adjuvant comprises a granulocyte/macrophage colony-stimulating factor (GM-CSF), a saponin QS21, a monophosphoryl lipid A, a CpG-containing oligonucleotide, or a bacterial toxin.

36. The method of any one of claims 25-35, further comprising co-administering with, prior to or following the administration of said recombinant Listeria an immune checkpoint protein inhibitor.

37. The method of claim 36, wherein said immune checkpoint protein is programmed cell death protein 1 (PD1), T cell membrane protein 3 (TIM3), adenosine A2a receptor (A2aR) and lymphocyte activation gene 3 (LAGS), killer immunoglobulin receptor (KIR) or cytotoxic T-lymphocyte antigen-4 (CTLA-4).

38. The method of any one of claim 25-35, or 25-36, further comprising co-administering a cytokine that enhances said anti-tumor immune response.

39. The method of claim 38, wherein said cytokine is: a type I interferon (IFN-α/IFN-(3), TNF-α, IL-1, IL-4, IL-12, INF-γ.

40. The method of any one of claims 25-39, wherein said method induces the expansion of T effector cells in peripheral lymphoid organs.

41. The method of claim 40, wherein said expansion of T effector cells leads to an increased ratio of T effector cells to regulatory T cells in the periphery without affecting the number of Tregs.

42. The method of claim 41, wherein said T effector cells are CD4+FoxP3- and CD8+ T-cells.

43. The method of claim 41, wherein said T effector cells are CD4+FoxP3− T cells.

44. The method of claim 41, wherein said regulatory T cells are CD4+FoxP+ T cells.

45. The method of any one of claims 1-24, wherein eliciting an anti-tumor T cell response in a subject having a tumor or cancer allows treating said tumor or cancer in said subject.

46. The method of any one of claims 25-44, wherein eliciting an anti-tumor T cell response in a subject having a tumor or cancer allows treating said tumor or cancer in said subject.
